(19) United States
(12) Patent Application Publication
(10) Pub. No.: US 2019/0008429 A1
(43) Pub. Date: Jan. 10, 2019

(54) INFORMATION ACQUIRING APPARATUS AND CONTROL METHOD

(71) Applicant: CANON KABUSHIKI KAISHA, Tokyo (JP)

(72) Inventor: Yohei YAMAMOTO, Hirosaki-shi (JP)

(73) Assignee: Canon Kabushiki Kaisha, Tokyo (JP)

(21) Appl. No.: 16/068,151

(22) PCT Filed: Jan. 31, 2017
(86) PCT No.: PCT/JP2017/003413

§ 371 Date: Jul. 5, 2018

(30) Foreign Application Priority Data
Feb. 8, 2016 (JP) 2016-021806

(51) Int. Cl. A61B 5/145; A61B 5/00; G01N 29/24; G01N 29/44

(52) U.S. Cl. CPC A61B 5/14542; A61B 5/0095; A61B 5/742; G01N 29/44; A61B 5/14546; G01N 29/2418; A61B 5/7257

(57) ABSTRACT

Provided is an information acquiring apparatus having: a calculating unit generating image data, based on signals acquired by transducers receiving an acoustic wave generated from an object by a plurality of times of light irradiation to the object; and a display controlling unit causing a display unit to display an image, wherein the calculating unit generates first image data using the signals corresponding to part of the plurality of times of light irradiation before the plurality of times of light irradiation complete, the display controlling unit causes the display unit to display an image based on the first image data before the plurality of times of light irradiation complete, the calculating unit generates second image data using the signals corresponding to more than the part of the plurality of times of light irradiation after the plurality of light irradiation complete

### TECHNICAL FIELD

The present invention relates to an information acquiring apparatus and a control method.

### BACKGROUND ART

Research on optical imaging apparatuses, which irradiate light from a light source (e.g. laser) onto an object (e.g. living body), and image the information in the object acquired based on the light which entered the object, is vigorously ongoing in medical fields. One optical imaging technique is photoacoustic imaging (PAI). In photoacoustic imaging, a pulsed light generated by a light source is irradiated to an object. Then a probe receives an acoustic wave (photoacoustic wave) which is generated by the object tissue, absorbing energy of the pulsed light which propagated and diffused in the object. The object information is imaged based on this received signal.

In photoacoustic imaging, the difference of absorptivity of optical energy between a target segment (e.g. tumor) and the other tissue is used. The test segment absorbs the irradiated optical energy and expands instantaneously. The elastic wave that is generated at this time is a photoacoustic wave. By mathematically analyzing this received signal, characteristic information (object information) inside the object can be acquired. The characteristic information is, for example, initial sound pressure distribution, optical energy absorption density distribution, and absorption coefficient distribution. The photoacoustic imaging can also be used for quantitative measurement of a specific substance in the object, and for oxygen saturation measurement in blood. Recently a pre-clinical study for imaging angiograms of small animals using this photoacoustic imaging and a clinical study applying this principle to the diagnosis of breast cancer and the like are actively ongoing.

A photoacoustic apparatus of PTL 1 uses a hemispherical probe in which a plurality of transducers are disposed. If this probe is used, the photoacoustic wave generated in a specific region can be received at high sensitivity. Therefore the resolution of the object information in this specific region increases. PTL 1 discloses that this probe scans on a plane, then the probe is moved in a direction perpendicular to this scanned plane, then scans on another plane, and this kind of scanning is repeated for a plurality of times. According to this method, object information having high resolution can be acquired over a wide range.

### CITATION LIST

### Patent Literature

### [PTL 1]

Japanese Patent Application Laid-Open No. 2012-179348

### SUMMARY OF INVENTION

### Technical Problem

The object information can be acquired by performing image reconstruction processing on the acoustic signals which a plurality of transducers received. The image reconstruction processing is, for example, backprojection in the time domain or Fourier domain, or is such data processing as phased addition processing, which is normally used for a tomographic technique. These processing operations normally require a large calculation amount. Therefore in some cases it is difficult to generate the object information following the reception of the acoustic wave by the probe. In concrete terms, imaging following the reception of an acoustic wave becomes difficult when high resolution of the image or high frequency of light irradiation is demanded.

With the foregoing in view, it is an object of the present invention to improve followability to the signal data acquisition when the object information is visualized in the photoacoustic measurement.

### Solution to Problem

The present invention uses an information acquiring apparatus, comprising:

a calculating unit configured to generate image data, based on signals acquired by transducers receiving an acoustic wave generated from an object by a plurality of times of light irradiation to the object; and

a display controlling unit configured to cause a display unit to display an image based on the image data, wherein

the calculating unit generates first image data using the signals corresponding to part of the plurality of times of light irradiation before the plurality of times of light irradiation complete,

the display controlling unit causes the display unit to display an image based on the first image data before the plurality of times of light irradiation complete,

the calculating unit generates second image data using the signals corresponding to more than the part of the plurality of times of light irradiation after the plurality of light irradiation complete, and

the display controlling unit causes the display unit to display an image based on the second image data after the plurality of times of light irradiation complete.

The present invention also uses a display method for an image generated based on signals acquired by transducers receiving an acoustic wave generated from an object by a plurality of time of light irradiation to the object,

the method comprising:

generating first image data using the signals corresponding to part of the plurality of times of light irradiation before the plurality of times of light irradiation complete, and causing a display unit to display an image based on the first image data; and

generating second image data using the signals corresponding to light irradiation more than the part of the plurality of times of light irradiation after the plurality of times of light irradiation complete, and causing the display unit to display an image based on the second image data.

### Advantageous Effects of Invention

According to the present invention, followability to the signal data acquisition can be improved when the object information is visualized in the photoacoustic measurement.

Further features of the present invention will become apparent from the following description of exemplary embodiments with reference to the attached drawings.

### BRIEF DESCRIPTION OF DRAWINGS

FIG. 1 is a schematic diagram depicting a configuration of an object information acquiring apparatus according to Embodiment 1.

FIG. 2 is a flow chart depicting an operation of the object information acquiring apparatus according to Embodiment 1.

FIG. 3 is a schematic diagram depicting the connection of the object information acquiring apparatus according to Embodiment 1.

FIGS. 4A and 4B are diagrams depicting an example of display data selection when the supporter performs linear motion.

FIGS. 5A and 5B are diagrams depicting an example of display data selection when the supporter performs spiral motion.

FIGS. 6A and 6B are diagrams depicting a modification of display data selection when the supporter performs spiral motion.

FIG. 7 is a schematic diagram depicting a configuration of an object information acquiring apparatus according to Embodiment 2.

FIG. 8 is a flow chart depicting an operation of the object information acquiring apparatus according to Embodiment 2.

FIG. 9 is a schematic diagram depicting a configuration of an object information acquiring apparatus according to Embodiment 3.

FIG. 10 is a flow chart depicting an operation of the object information acquiring apparatus according to Embodiment 3.

### DESCRIPTION OF EMBODIMENTS

Embodiments of the present invention will be described with reference to the drawings. Dimensions, materials, shapes, relative positions, and the like, of the elements described below should be appropriately changed depending on the configuration and various conditions of the apparatus to which the present invention is applied. Therefore the scope of the present invention is not limited to the following description.

The present invention relates to a technique to detect an acoustic wave propagated from an object, generate characteristic information inside the object, and acquire the generated information. Therefore the present invention is regarded as an object information acquiring apparatus or a control method thereof, an object information acquiring method and a signal processing method, or a display method. The present invention is also regarded as a program that causes an information processing apparatus, which includes such hardware resources as a CPU and memory, to execute these methods, or a storage medium storing this program.

The object information acquiring apparatus of the present invention includes an apparatus utilizing a photoacoustic effect, which irradiates light (electromagnetic wave) to an object, receives an acoustic wave generated inside the object, and acquires the characteristic information of the object as image data. In this case, the characteristic information is information on characteristic values corresponding to each of the plurality of positions inside the object, and this information is generated by using the receive signals acquired by receiving the photoacoustic wave.

The characteristic information acquired by the photoacoustic measurement is values reflecting the absorptivity of optical energy. For example, the characteristic information includes a generation source of the acoustic wave generated by the light irradiation, an initial sound pressure inside the object, an optical energy absorption density or absorption coefficient derived from the initial sound pressure, and a concentration of a substance constituting the tissue. For the substance concentration, oxygen saturation distribution may be calculated by determining oxyhemoglobin concentration and deoxyhemoglobin concentration. Glucose concentration, collagen concentration, melanin concentration, volume fraction of fat or water and the like may be determined.

Based on the characteristic information at each position in the object, a two-dimensional or three-dimensional characteristic information distribution is acquired. The distribution data can be generated as image data. The characteristic information may be determined, not as numeric data, but as distribution information at each position in the object. In other words, such distribution information as the initial sound pressure distribution, energy absorption density distribution, absorption coefficient distribution, and oxygen saturation distribution may be determined. The three-dimensional (or two-dimensional) image data is the distribution of characteristic information on reconstruction units disposed in a three-dimensional (or two-dimensional) space. The reconstruction units are voxels in the case of three-dimensional space, and pixels in the case of two-dimensional space.

The acoustic wave referred to in the present invention is typically an ultrasonic wave, including an elastic wave that is called a sound wave or an acoustic wave. An electric signal converted from an acoustic wave by a probe or the like is also called an acoustic signal. In this description, the use of the phrase “ultrasonic wave” or “acoustic wave” is not intended to limit the wavelength of the elastic waves. An acoustic wave generated by the photoacoustic effect is also called a photoacoustic wave or a light-induced ultrasonic wave. An electric signal originating in a photoacoustic wave is also called a photoacoustic signal.

### Embodiment 1

(Apparatus Configuration)

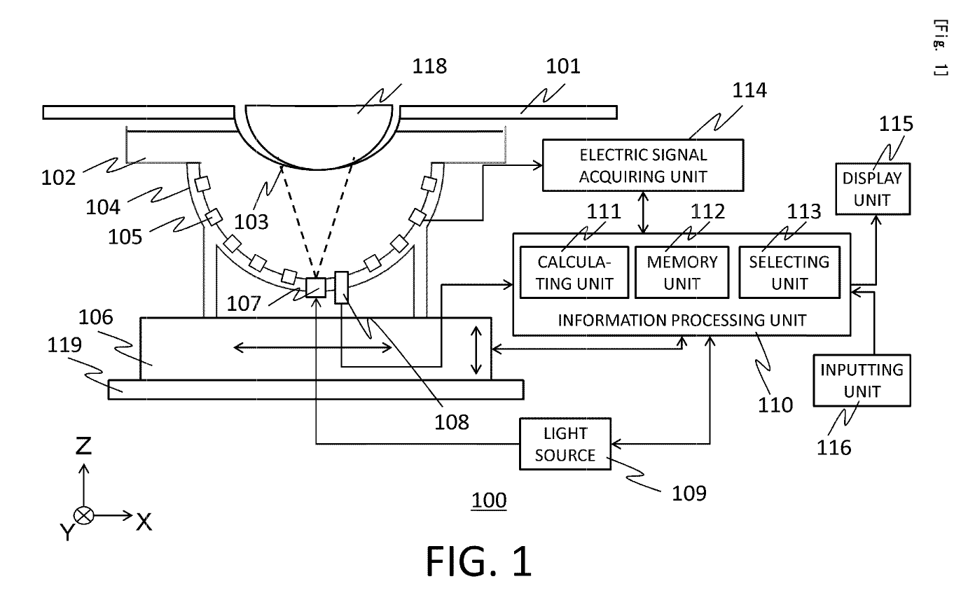

FIG. 1 is a schematic diagram depicting a configuration of an object information acquiring apparatus 100 according to Embodiment 1.

A test object 118 is a target of the measurement. Examples are body parts, such as a breast, a hand and a leg, and a phantom which stimulates the acoustic characteristics and optical characteristics of a living body, and is used for adjusting the apparatus. In concrete terms, the acoustic characteristics are the propagation speed and damping rate of the acoustic wave, and the optical characteristics are the absorption coefficient and scattering coefficient of the light. A light absorber is a substance which exists inside the test object 118, and which has a large light absorption coefficient with respect to the light irradiated from the light source 109. In the case of a living body, the light absorber is, for example, hemoglobin, water, melanin, collagen or lipid. In the case of a phantom, the phantom includes a substance having desired optical characteristics.

The light source 109 is an apparatus that can irradiate a pulsed light for a plurality of times. For the light source, a laser is preferable because of its high power, but the light source may be a light emitting diode, a flash lamp or the like. To effectively generate a photoacoustic wave, it is desirable that the light source 109 can irradiate the pulsed light for a plurality of times at sufficiently short intervals, in accordance with the thermal characteristics of the object. In the case when the object is a living body, the pulse width of the pulsed light generated from the light source 109 is preferably several tens nanoseconds or less. The wavelength of the pulsed light is preferably about 700 nm to 1200 nm, which is a near-infrared region called a biological window. The light in this region reaches a relatively deep portion of a living body, hence information on the deep portion of the living body can be acquired. If the measurement is limited to the surface portion of a living body, light having a 500 nm to 700 nm wavelength, which is visible light to a near-infrared region, may be used. The wavelength of the pulsed light preferably has a high absorption coefficient with respect to the observation target.

A holding unit 103 is installed in an opening of a support table 101 to support the object, so as to hold the test object 118, which is a part of the object inserted through the opening, and to maintain the shape of the test object 118 in a constant state. In the case of providing a plurality of shape holding units 103, which are selectable according to the shape of the test object 118, an installing unit to replace the shape holding units 103 is disposed at the opening of the support table 101. If a material having an acoustic impedance close to that of the object is selected as the material of the holding unit 103, the reflection of the acoustic wave on the interface between the test object 118 and the holding unit 103 can be reduced. The thickness of the holding unit 103 is preferably thin so as to reduce reflection of the acoustic wave by the holding unit 103. In the case of irradiating light to the test object 118 via the holding unit 103, it is preferable that the holding unit 103 has high transmittance of the light. For example, polymethyl pentene, polyethylene terephthalate, polycarbonate and the like can be used for the holding unit 103. If the test object 118 is a breast, a holding unit having a shape of a sphere, that is sectioned by a certain cross-section, should be used so as to minimize deformation of the breast. For the holding unit 103, a sheet type film, a rubber sheet or the like may be used instead of the above mentioned members. The test object 118 may be measured without using the holding unit 103.

An optical system 107 transmits the pulsed light generated by a light source 109. For example, the optical system 107 includes such optical apparatuses as a lens, mirror, prism, optical fiber and diffusion plate. When the light is guided, the shape and light density may be changed using these optical apparatuses so that a desired light distribution is generated. As a standard related to irradiation of a laser beam or the like to a biological tissue, the intensity of light that can be irradiated to a unit area (maximum permissible exposure) has been specified. To satisfy this standard, it is preferable to spread the light over a certain surface area, as indicated by the broken line in FIG. 1.

It is preferable that the optical system 107 includes an optical mechanism (not illustrated) which detects irradiation of the pulsed light to the test object 118, and generates synchronization signals used for receiving and storing photoacoustic waves. For example, a part of the pulsed light generated by the light source 109 is split and guided to the photosensor via an optical system, such as a half mirror, and is detected by an output signal of a photosensor. In the case of using a fiber bundle for guiding the pulsed light, a part of the fibers are branched to guide the light to the photosensor. The synchronization signal generated by this detection is output to an electric signal acquiring unit 114 and an information processing unit 110.

A transducer 105 detects a photoacoustic wave that is generated by the light that is irradiated to the test object 118, and outputs an electric signal. It is preferable that the transducer has a high reception sensitivity and wide frequency band with respect to the photoacoustic wave from the test object 118. As a member constituting the transducer 105, a piezoelectric ceramic material represented by PZT (lead zirconate titanate) or a polymer piezoelectric film material represented by PVDF (polyvinylidene fluoride), for example, can be used. Further, an electrostatic capacitance type element, such as CMUT (capacitive micro-machined ultrasonic transducer) or a transducer using a Fabry-Perot interferometer can also be used.

A supporter 104 supports the transducer 105. In this example, an approximately hemispherical container is used as the supporter. A plurality of transducers 105 are installed inside the hemispherical container, and an output end of the optical system 107 is installed in the base portion. An acoustic matching material 102 is filled into the container of the supporter 104. To support these members, the material of the supporter 104 is preferably, for instance, a metal having strong mechanical strength.

Each of the plurality of transducers 105 installed in the supporter 104 is disposed so that the direction, in which sensitivity of reception directivity is the highest (directional axis), is directed toward a specific region. The specific region is, for example, a center of the curvature of the supporter. By such an arrangement of the transducers 105, a region where the acoustic wave is received at high sensitivity and resolution if the generated image is high (high sensitivity region) is formed. The high sensitivity region can be defined, for example, as a region having a resolution that is at least half the maximum resolution, centering around the point at which the resolution is the highest.

If a desired high sensitivity region can be formed, the arrangement of the transducers and shape of the supporter are not limited to the above description. It is sufficient if at least a part of the elements of the plurality of transducers 105 are disposed in the supporter 104, so as to receive the photoacoustic waves generated in the high sensitivity region at high sensitivity. Further, it is sufficient if the plurality of transducers 105 are disposed in the supporter 104 so that the directive axes of the transducers concentrate, instead of disposing the directive axes of the transducers 105 in parallel. For the supporter 104, instead of the hemispherical shape, various other shapes can be used, such as a partial ellipsoid, a cup, a bowl and a combination of planes and curved surfaces. It is preferable that the plurality of transducers 105 are disposed on the supporter 104, so that the high sensitivity region, which is determined by the arrangement of the plurality of transducers 105, is formed at a position where placement of the test object 118 is expected. If there is a holding unit 103 which holds the shape of the test object 118, it is preferable to form the high sensitivity region near the holding unit 103.

A scanning stage 106 is disposed on a stage base 119. The scanning stage 106 changes a relative position of the supporter 104 with respect to the test object 118 in the X, Y and Z directions in FIG. 1. The scanning stage 106 includes a guide mechanism in the X, Y and Z directions, a drive mechanism in the X, Y and Z directions, and a position sensor to detect a position of the supporter in the X, Y and Z directions, which are not illustrated. The supporter 104 is disposed on the scanning stage 106 as illustrated in FIG. 1. This means that the guide mechanism is preferably a linear guide or the like which can withstand a heavy load. For the driving mechanism, a lead screw mechanism, a link mechanism, a gear mechanism, a hydraulic mechanism or the like can be used. For the driving force, a motor, for example, can be used. For the position sensor, an optical or magnetic encoder, for example, can be used. The scanning stage 106 corresponds to the moving unit of the present invention.

The electric signal acquiring unit 114 collects electric signals from the plurality of transducers 105 in a time-series. Typically the electric signal acquiring unit 114 is constituted by such elements as a CPU, an OP amp and an A/D converter, and such circuits as an FPGA and an ASIC. The electric signal acquiring unit 114 generates digital signals by performing filtering, amplification and A/D conversion on the analog signals received from a plurality of transducers 105, and transfers the generated digital signals to the information processing unit 110. The electric signal acquiring unit 114 may be constituted by a plurality of elements and circuits.

The acoustic matching material 102 fills the space between the test object 118 and the holding unit 103, and the space between the holding unit 103 and the transducers 105, so as to acoustically bond the test object 118 and the transducers 105. The material of the acoustic matching material 102 in each space may be different. The acoustic matching material 102 is preferably a material of which acoustic impedance is close to those of the test object 118 and the transducers 105, and for which the attenuation of the acoustic wave is small. It is also preferable that the acoustic matching material 102 transmits the pulsed light. For example, water, castor oil, gel or the like can be used as the acoustic matching material 102.

An imaging element 108 images the test object 118 and outputs the signal to the information processing unit 110. The information processing unit 110 analyzes the signal output from the imaging element 108, and generates imaging data. For the imaging element 108, an optical imaging element, such as a CCD sensor or a CMOS sensor can be used. For the imaging element 108, a piezoelectric element, CMUT or the like may be used. In the case of the latter, a part of the elements of the plurality of transducers 105 may be used as the imaging element 108. The imaging element 108 is not limited to the above description, as long as the test object 118 can be imaged. An image processing unit for the imaging element 108 may be disposed as well. The imaging element 108 may be disposed in any position as long as the test object 118 can be imaged.

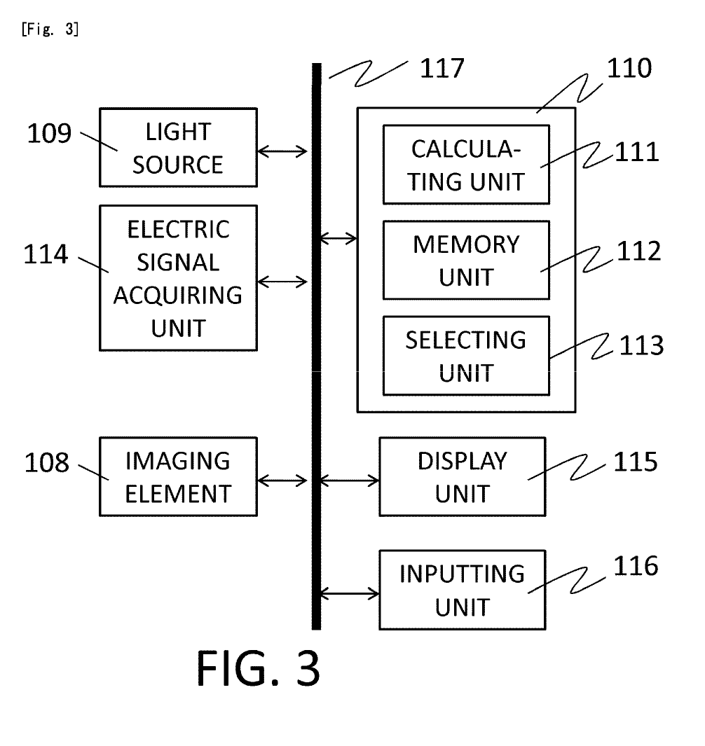

The information processing unit 110 includes a calculating unit 111, a memory unit 112, and a selecting unit 113. The calculating unit 111 is typically constituted by such elements as a CPU, a GPU and an A/D converter, and such circuits as an FPGA and an ASIC. The calculating unit 111 performs signal processing on an electric signal output from the electric signal acquiring unit 114, and acquires characteristic information inside the test object 118. The calculating unit 111 also controls operation of each composing element constituting the object information acquiring apparatus via a bus 117, as depicted in FIG. 3. By using the information processing unit 110 that can pipe-line process a plurality of signals simultaneously, the object information acquiring time can be decreased.

The memory unit 112 stores received signals by the plurality of transducers 105, which were output from the electric signal acquiring unit 114 as digital signals. The memory unit 112 is typically constituted by a ROM, a RAM or such a storage medium as a hard disk. The memory unit 112 may be constituted not by one storage medium, but by a plurality of storage media. A non-volatile storage medium of the memory unit 112 can store programs which the calculating unit 111 executes.

The selecting unit 113 selects a received signal (visualization target) from which the calculating unit 111 acquires information inside the test object 118. The selecting unit 113 is constituted by such elements as a CPU, a comparator, a counter and an A/D converter, and such circuits as an FPGA and an ASIC. The calculating unit 111 may perform the operation of the selecting unit 113. The selecting unit 113 may be installed separately from the information processing unit 110. The information processing unit 110, the calculating unit 111 and the selecting unit 113 can be installed in an information processing apparatus, such as a PC and a workstation.

A display unit 115 displays information on the test object 118 which is output from the information processing unit 110 as a distribution image, numeric data or the like. For example, a liquid crystal display, plasma display, organic EL display, FED or the like can be used for the display unit 115. The display unit 115 may be provided separately from the object information acquiring apparatus of the present invention. In this case, the optical information acquiring apparatus outputs image data which indicates the characteristic information, and performs display control. The information processing unit 110 (particularly the calculating unit 111) functions as the display controlling unit of the present invention regardless of whether the display unit 115 is included in the object information acquiring apparatus or not.

An inputting unit 116 is a user interface that can receive input information from the user. The user specifies desired information to the information processing unit 110 using the inputting unit 116. For the inputting unit 116, a keyboard, a mouse, a dial, a push button, a touch panel or the like can be used. In the case of using a touch panel, the display unit 115 may play the role of the inputting unit 116 as well. Any user interface may be used for the inputting unit 116, as long as the information input from the user can be received. The inputting unit 116 may be provided separately from the object information acquiring apparatus of the present invention. In the case of using a PC or workstation as the information processing unit 110, the user interface function of the PC can be used as the display unit 115 and the inputting unit 116.

(Processing Flow)

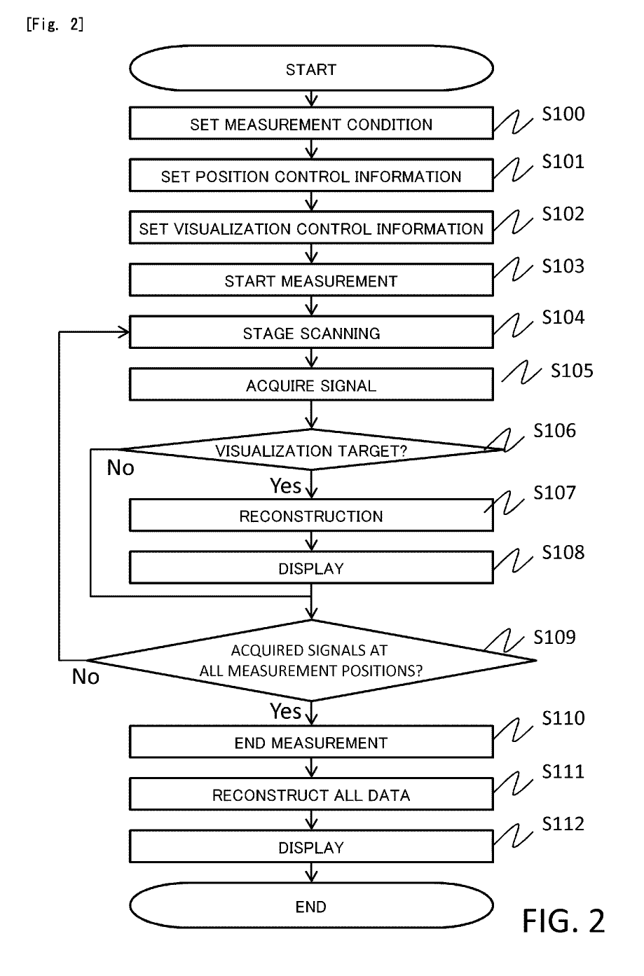

FIG. 2 is a flow chart of the operation according to Embodiment 1. In this flow, display control having high followability to the signal acquisition is performed in the first half portion (steps S100 to S109). Therefore the first half portion is suitable for sequential display, which uses a relatively small amount of data, and is performed in parallel with light irradiation and acoustic wave reception. In the sequential display, first image data is generated using electric signals corresponding to part of a plurality of times of light irradiation. Typically in the first half portion, an image of the object is gradually displayed as the support moves. In other words, in the sequential display, an image of the object is generated and displayed before all the light irradiation is complete. The latter half portion (steps S110 to S112), on the other hand, is suitable for a high definition display method after the scanning ends, which uses more data than the sequential display. In the high definition display, second image data is generated using electric signals corresponding to light irradiation more than the part of a plurality of times of light irradiation used for the first image data. In this description, the sequential display, in which the first image data is generated, is also called the first display; and the high definition display, in which the second image data is generated, is also called the second display.

In step S100, measurement conditions are set. For example, based on the information received from the user, the information processing unit 110 performs settings concerning the information on the test object 118, type of the holding unit 103, region of interest and the like. The measurement conditions may be stored in the memory unit 112 in advance, so that the conditions are set based on the selection by the user via the inputting unit 116. The ID information of the equipment connected to the apparatus may be read so that the measurement conditions are set based on this read information.

In step S101, the position control information of the scanning stage 106 is set based on the measurement conditions which were set in S100. In concrete terms, the information processing unit 110 calculates the moving region S of the scanning stage 106, light emitting timing, light irradiation position, and photoacoustic wave receiving position, based on the measurement conditions which were set in S100. At this time, the moving path, scanning speed, acceleration profile and the like may also be set. The receiving position is the position of the supporter 104 when the light source 109 emits the light.

The position and size of the high sensitivity region G are determined based on the arrangement of the plurality of transducers 105. Therefore based on the region of interest and the arrangement information of the plurality of transducers 105 on the supporter 104, the calculating unit 111 sets the moving region S so that the high sensitivity region G is formed inside the region of interest. If a plurality of holding units having different sizes are included, the moving region S may be determined based on the size information of the holding units 103 and arrangement information of the transducers 105. The moving region S may also be determined based on the image data captured by the imaging element 108 and the arrangement information of the transducers 105.

Further, information on the moving region S corresponding to the high sensitivity region, a region of interest, a holding unit 103 and the like, and the light emitting timing, the light irradiation position, and the photoacoustic wave receiving position, may be stored in the memory unit 112 in advance. The user may set the arbitrary moving region S, light emitting timing, light irradiation position and photoacoustic wave receiving position using the inputting unit 116. It is preferable that the driving of the light source 109 and scanning stage 106 is controlled so that overlapping of the high sensitivity regions G between the first signal acquiring position and the second signal acquiring position becomes a desired degree of overlapping.

In this embodiment, the high sensitivity region G has a spherical shape, hence it is preferable to acquire a signal at least once until the supporter 104 moves for a same distance as the radius of the high sensitivity region G. The resolution can be uniform as the distance of moving the supporter 104 from the first pulsed light irradiation to the second pulsed light irradiation shortens. However, if the moving distance is short (that is, if the moving speed is slow), it takes time to acquire all the signals. Therefore it is preferable to appropriately set the moving speed and intervals of the received signal acquisition timings, considering the desired resolution and measurement time. The resolution and measurement time should be set based on the input values and selected conditions via the inputting unit. For example, if the user wants to decrease the measurement time, the moving speed is increased and a number of receiving positions is decreased. If a certain level of high resolution is demanded even in the sequential display, a higher number of receiving positions are set.

In step S102, information on the visualization target, out of the signals received at the photoacoustic wave receiving positions which were set in S101, is set. In the sequential display mode, display is performed in parallel with the pulsed light irradiation and acoustic wave reception, hence followability to the scanning is high, but data amount that can be processed is low because the processing performance is low. Therefore data to be the processing target in this step is limited.

The calculating unit 111 calculates the visualization target receiving position, and sets the selecting unit 113 based on the measurement conditions, the control information and arrangement information of the plurality of transducers 105 on the supporter 104, which were set in S100 and S101. Or a number of times of pulsed light irradiation at a visualization target receiving position may be calculated, whereby the selecting unit 113 is set. The visualization target receiving position or information on a number of times of pulsed light irradiation may be stored in the memory unit 112 in advance. Or the user may input the visualization target receiving position or a number of times of irradiation using the inputting unit 116, and output this information to the information processing unit 110, whereby the selecting unit 113 is set.

To implement the sequential display, it is necessary to complete the image reconstruction processing and display of the first visualization target signals while the support moves from the first visualization target position to the second visualization target position. Visualization target selection is implemented on the basis of such necessity. Signals that are not the visualization targets may be acquired between the first visualization target position and the second visualization target position. These signals can be stored and used for final high definition display.

((Raster Scan))

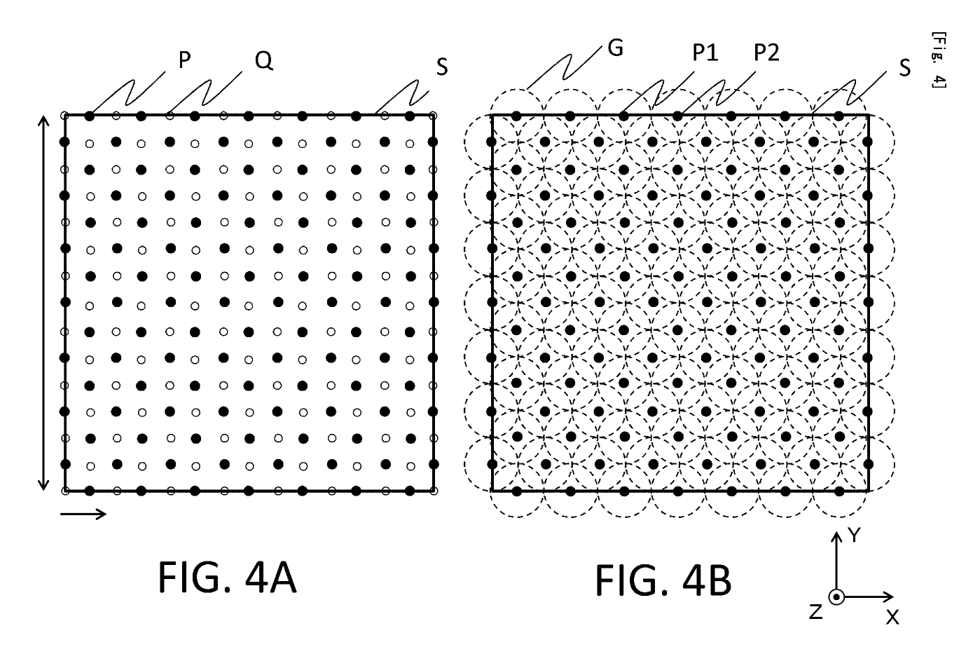

FIG. 4 is an example of selecting the visualization target data in the case when the supporter 104 performs a raster scan constituted by a linear motion and direction change. The supporter 104 acquires the photoacoustic wave at predetermined receiving positions while moving in the X direction, moves one step in the Y direction, and then changes the direction. In FIG. 4A, P (black dot) and Q (white dot) indicate the photoacoustic wave receiving positions in the moving region S. The photoacoustic wave received at the receiving position P is a visualization target, and the photoacoustic wave received at the receiving position Q is not a visualization target. By limiting the processing targets like this, an image can be generated even within a limited time in the sequential display.

FIG. 4B is a diagram generated by extracting only the receiving positions P and overlapping the high sensitivity regions G of the supporter 104 at each receiving position. To display an image that is as good as possible within the range allowed by the processing performance in the sequential display, it is preferable that each region, on which the high sensitivity regions G are overlapped, fills the region of interest as much as possible. For this, it is preferable to set the visualization control information so that the high sensitivity regions G overlap, or no gap is generated between the high sensitivity regions G in an area between the first visualization target receiving position (P1) and the second visualization target receiving position (P2), as illustrated in FIG. 4B. Further, the receiving positions for the sequential display may be arranged at even or approximately even spatial positions. Thereby the image quality of the image to be displayed becomes spatially uniform, and a drop in diagnostic performance due to the generation of locally different image quality can be suppressed. Here “approximately even” refers to the case when each distance between the receiving positions is either the same or in a range of positions where resolution of the sequential display image drops 10% or less from the maximum resolution.

The high sensitivity region G of the present embodiment is spherical, hence it is preferable that the signal is visualized at least once while the supporter 104 moves for a distance the same as the radius of the high sensitivity region G. If one high sensitivity region G is made bigger, a sequential display without gaps can be implemented even if the number of times of signal acquisition is low. In this case, however, image definition drops in the high sensitivity region G. Therefore it is preferable to adjust the control parameters in accordance with the desired image quality, scanning speed (that is, measurement time) and capability of the electric signal acquiring unit in the sequential display.

((Spiral Scan))

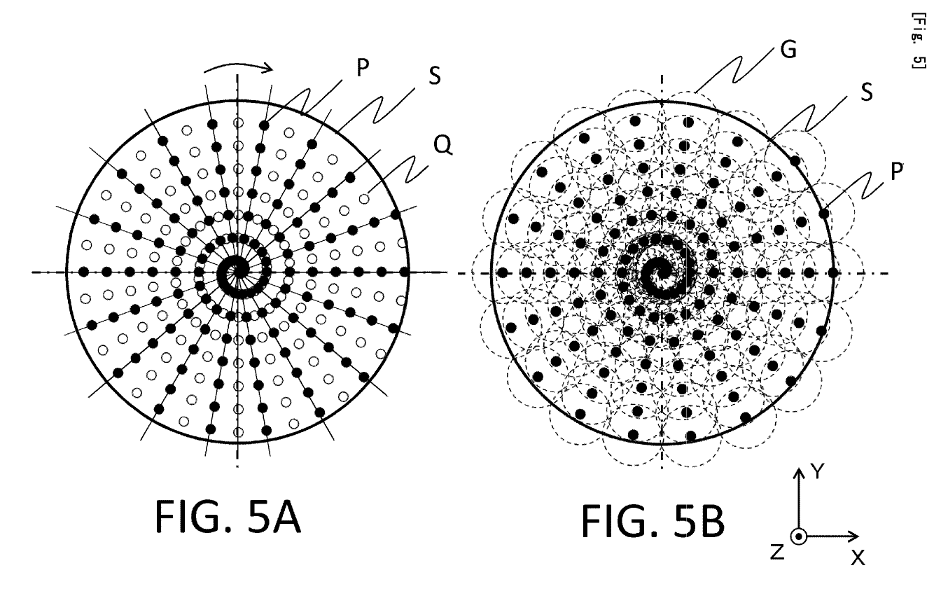

FIG. 5 illustrates an example of selecting the visualization target data in the case when the supporter 104 performs a spiral motion. As mentioned above, the space between the holding unit 103 and supporter 104 is filled with the acoustic matching material 102. In the case of the spiral motion in which the locus of the center of the support is a smooth curved line, the change of the force applied to the acoustic matching material 102 in the circumferential direction is smooth. As a result, the generation of factors to interrupt propagation of the photoacoustic waves, such as waves and bubbles, can be suppressed.

FIG. 5A indicates the photoacoustic wave receiving positions P and Q in the moving region S. The received signals at specific angles with respect to the center of the moving region S are set as the visualization targets. Thereby the image update position, when the display unit 115 refreshes, can be made constant. The received signals may be selected based on the coordinate positions, instead of the angle settings. FIG. 5B depicts the state of extracting the photoacoustic wave receiving positions P to be visualized, and indicates the range of each high sensitivity region G corresponding to each receiving position P. Even in the sequential display, it is preferable to set the visualization control information so that the overlapped regions of the high sensitivity regions G cover the entire moving region S. However, the visualization control information should be appropriately changed in accordance with the information processing capability and size of the high sensitivity region G. For example, if the high sensitivity region G is relatively large, the calculation amount is high, hence it is preferable to set the conditions to save the calculation resources, such as increasing the voxel size.

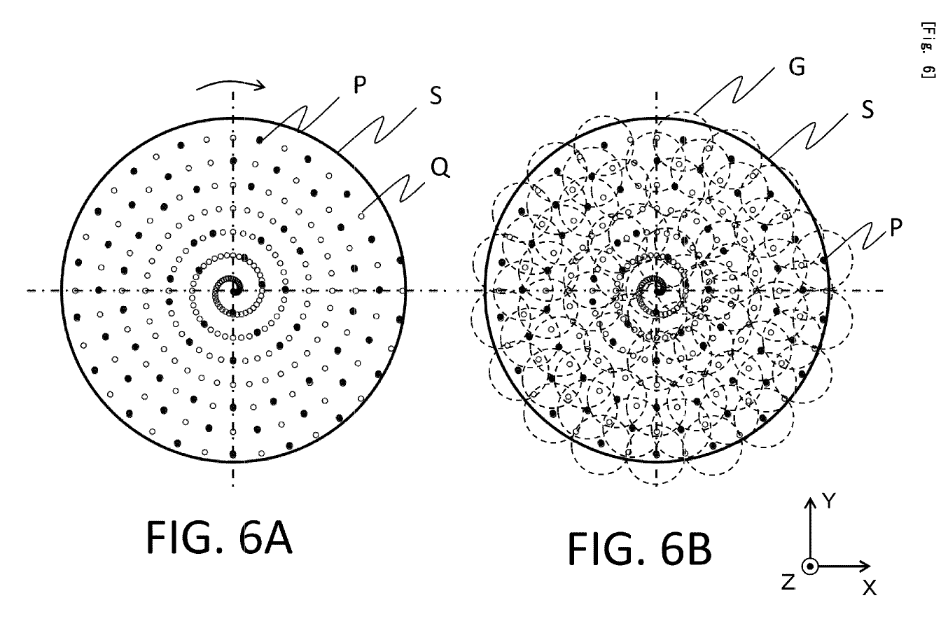

FIG. 6 illustrates a modification of the data selection when the spiral motion is performed. FIG. 6A indicates the photoacoustic wave receiving positions P and Q in the moving region S. FIG. 6B also indicates the high sensitivity region G at the photoacoustic wave receiving position P to be visualized. As illustrated in FIG. 6B, it is preferable to set the visualization control information so that the overlapping of the high sensitivity regions G become less between the receiving position of the first visualization target and the receiving position of the second visualization target. For example, in each high sensitivity region G, the portion overlapping with other high sensitivity regions G is 50% or less, preferably 30% or less. It is also preferable that the receiving position selection patterns, to minimize the overlapped regions, are stored in memory or the like in advance.

If the distance from the first visualization target receiving position to the second visualization target receiving position is increased, the time to be used for image reconstruction increases and followability to the signal data acquisition improves. If the distance from the first visualization target receiving position to the second visualization target receiving position is decreased, on the other hand, the time to be used for image reconstruction decreases, but resolution becomes uniform. Therefore the intervals of the visualization target receiving positions are appropriately set considering the balance between the desired resolution and the image reconstruction processing capability. For example, if the image reconstruction capability is relatively high, the information amount to be used for reconstruction may be increased by increasing the number of receiving positions. Further, if the image reconstruction is relatively high, the resolution may be improved by making the pitch of the reconstruction units denser.

The moving path of the support is not limited to the raster scan and spiral scan. As illustrated in FIG. 6, the receiving positions P and Q need not be arranged alternately. It is preferable that the receiving positions P and Q are arranged in accordance with the information processing speed, and converge the high sensitivity regions G in the region of interest. In FIG. 4 to FIG. 6, the receiving positions P and Q are indicated as clear dots. However, the present invention is not limited to the method in which the support repeats moving and stopping, and the photoacoustic measurement is performed when the support is stopped (step and repeat). The present invention can also be applied to a method of performing the photoacoustic measurement while the support is moving (continuous scanning). In the case of continuous scanning as well, the information inside the object can be reconstructed based on such information as the moving speed of the support, positions at which the light was irradiated, and positions at which the acoustic wave reception was started and stopped. In the case of continuous scanning, the object image can be reconstructed regarding the receiving positions P and Q as the center position of the support when the pulsed light was irradiated, center position of the support when the acoustic wave reception was started, a characteristic position during the acoustic wave reception and the like.

In step S103, insertion of the test object 118 into the holding unit 103 is confirmed and measurement is started.

In step S104, the supporter 104 is moved to the receiving positions P and Q in the moving region that were set in S101. The scanning stage 106 sequentially sends the coordinate information of the supporter 104 to the information processing unit 110.

In step S105, the light source 109 irradiates the pulsed light and generates the photoacoustic wave from the light absorber inside the test object 118. The plurality of transducers 105 receive the acoustic wave propagated through the acoustic matching material 102. The electric signal acquiring unit 114 performs amplification and digitization on the analog signals output from the transducers 105, and outputs the digitized signals. The information processing unit 110 associates the digital electric signals with the coordinate positions of the support in S104, and saves this information in the memory unit 112. The associating method is arbitrary. For example, the light source may send a number of times of pulsed light irradiation to the information processing unit 110, and the information may be stored in the memory unit 112. Or a number of times of pulsed light irradiation counted by the information processing unit 110 may be stored in the memory unit 112, and may be saved as an electric signal associated with the number of times of irradiation in S105. The method is not limited to the above method only if the electric signal can be associated with the pulsed light, which is irradiated for a multiple number of times.

In step S106, it is determined whether the received signals saved in S105 are visualization targets which were set in S102. For example, if “visualization target receiving positions” are set in the selecting unit 113, the selecting unit 113 compares the coordinate position of the support in S104 with the setting information. If “a number of times of pulsed light irradiation” is set in the selecting unit 113, the selecting unit 113 compares the number of times of irradiation in S105 with the setting information. If the received signal is not a visualization target (NO in S106), processing advances to S109. If the received signal is a visualization target (YES in S106), processing advances to S107.

In step S107, the image reconstruction is performed on the visualization target received signals, whereby the information inside the test object 118 is acquired. For the image reconstruction algorithm, for instance, backprojection in the time domain or Fourier domain, or an inverse problem analysis method using repeat processing, which is used for a tomographic technique, can be used. In this case, the later mentioned S109 and S104 to S106 may be executed in parallel.

In this step corresponding to the sequential display, processing with a high calculation amount is not always necessary. For example, even in the case of generating the final image data by repeat processing, a method which requires a less calculation amount may be used in this step. Further, in this step, instead of displaying the absorption coefficient distribution that requires calculation based on the light quantity distribution, an initial sound pressure distribution, which can be acquired by a simple reconstruction or an optical energy absorption density distribution that can be acquired using the Gruneisen coefficient which has a predetermined value for each object, may be displayed. In this step, one process of reconstruction may be performed based on electric signals corresponding to a plurality of receiving positions.

In this step S108, the information inside the test object 118 acquired in S107 is displayed on the display unit 115. The display method here is a sequential display. In this case, it is preferable that images corresponding to the high sensitivity regions are gradually added and the image expands as the scanning progresses. In other words, an image of the high sensitivity region centering around the position, at which the visualization target received signal is acquired, is sequentially added to the currently displayed image.

In step S109, it is determined whether the electric signals were received at all the receiving positions P and Q in the moving region S which was set in S101. If not acquired (NO in S109), the supporter 104 is moved to a second receiving position, which is different from the first receiving position in the moving region S (S104), and signals are acquired at the second receiving position (S105). Hereafter, the same step is repeated until electric signals are acquired at all the receiving positions in the moving region S which were set in S101. When the electric signals are acquired at all the receiving positions (YES in S109), processing advances to step S110, and measurement ends.

In step S111, the image reconstruction is performed for the received signals acquired in S103 to S110, and the characteristic information inside the test object 118 is acquired. In S111, data corresponding to more pulsed light beams than the case of generating one sequential display image is selected, and image data for high definition display is generated. Typically the image reconstruction is performed using all the received signals, including the signals at the receiving positions Q, stored in the information processing unit 110. However, a high definition display can be implemented by using more signals than the case of the sequential display, even if all the data is not used. In other words, in the high definition display, image data is generated using a higher total amount of electric signal data for image generation, compared with the case of the sequential display. Even in the case of repeatedly using the same electric signals as the electric signals used for the sequential display, an image based on more electric signals than the case of the sequential display can be generated.

The image reconstruction processing in S111 need not be executed immediately after acquiring the electric signals at all the receiving positions in the moving region S which were set (immediately after step S110). All the acquired data may be transferred to such an external storage apparatus as an HDD and flash memory or to a server, so that the reconstruction processing is performed any time or place desired by the user. Hence in this step, the reconstruction method with a high calculation amount can be used, unlike step S108. In step S111, the data generated in step S107 may be reused. In this case, the conditions, such as the pitch of the reconstruction units (e.g. pixel, voxel) must be adjusted to be consistent.

In step S112, the high definition characteristic information image generated in S111 is displayed on the display unit 115.

As described above, in this embodiment, a part of all the signals received at photoacoustic wave receiving positions in the moving region S of the scanning stage 106 are selected as the visualization targets in the sequential display. In other words, electric signals corresponding to the partial pulsed light beams are used. Thereby the followability to the signal data acquisition, when the object information is visualized, improves. If the final display image is the high definition image, received signals corresponding to more pulsed light beams than the received signals used for generating one sequential display image can be used (typically all the signals can be used). In other words, electric signals corresponding to more pulsed light beams than the above mentioned partial pulsed light beams are used.

### Embodiment 2

Embodiment 2 will be described focusing on aspects that are different from Embodiment 1.

### (Apparatus Configuration)

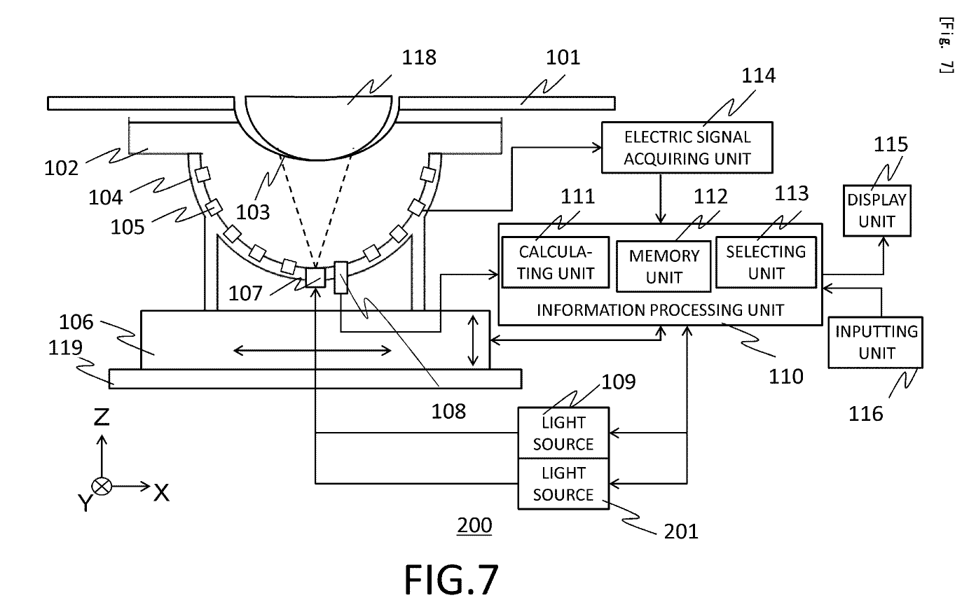

FIG. 7 is a schematic diagram depicting an object information acquiring apparatus 200 according to Embodiment 2. Embodiment 2 includes a plurality of light sources (109, 201), which generate pulsed light beams having mutually different wavelengths. By irradiating pulsed light beams having a plurality of wavelengths respectively, a concentration of substances or the like in the test object 118 can be calculated. For example, oxyhemoglobin concentration distribution, deoxyhemoglobin concentration distribution, oxygen saturation distribution and the like can be calculated.

A light source 201 is an apparatus configured to generate a pulsed light having a wavelength that is different from the light source 109. In the case of determining oxygen saturation, it is preferable to use two types of light, such as light of which wavelength is about 750 nm, and light of which wavelength is about 800 nm, in order to utilize the difference of light absorption spectrums between oxyhemoglobin and deoxyhemoglobin. The light source 109 and the light source 201 alternately irradiate the pulsed light beams having mutually different wavelengths to the test object 118. In the case of forming the light irradiation lines by alternate irradiation of the first wavelength and the second wavelength which is different from the first wavelength as a set in this way, the measurement time is decreased compared with the case of performing a plurality of times of measurement for each wavelength. Instead of using a plurality of light sources, a light source, which can switch the wavelength to generate (e.g. wavelength variable laser), may be used.

(Processing Flow)

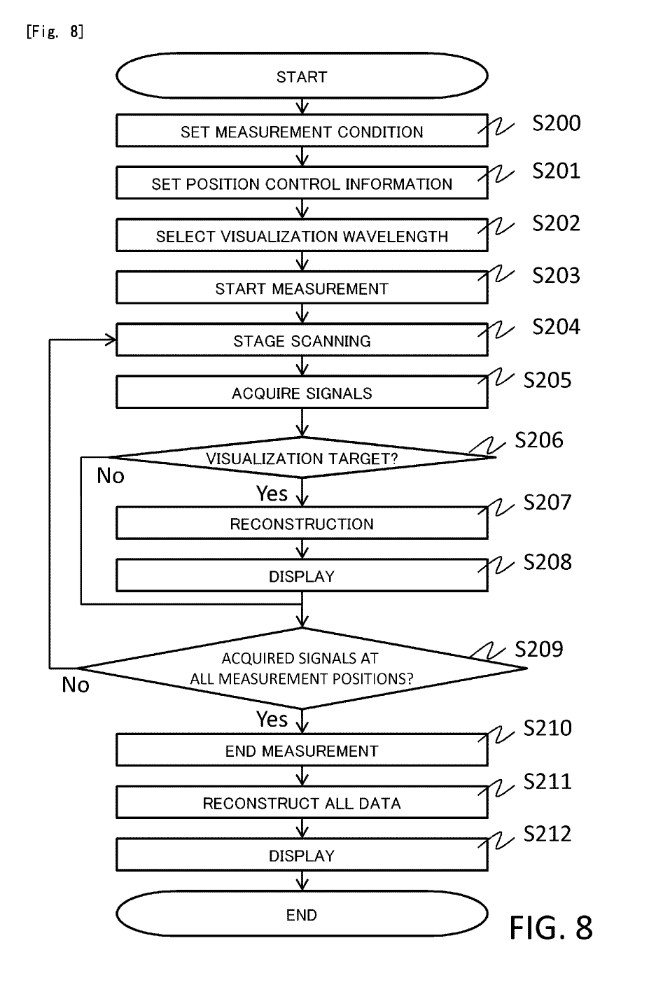

FIG. 8 is a flow chart of the operation according to Embodiment 2.

Steps S200 and S201 are the same as S100 and S101 of Embodiment 1.

In step S202, visualization target information is set from the signals received at photoacoustic wave receiving positions which were set in S201. In this case, the calculating unit 111 calculates the visualization target receiving positions based on the wavelength of the light source, and sets the selecting unit 113. The calculating unit 111 may calculate a number of times of pulsed light irradiation at the visualization target receiving positions and set the selecting unit 113. The information on the visualization target receiving positions or the number of times of pulsed light irradiation may be stored in the memory unit 112 in advance. Or the visualization target receiving positions or the number of times of irradiation may be calculated by the user inputting the visualization target wavelengths using the inputting unit 116, and outputting this information to the information processing unit 110.

If the wavelength, of which absorption coefficient by the measurement target is higher, is selected as the visualization target when the sequential display is performed, an image having high resolution can be acquired. If a longer wavelength is selected, a deep portion of the measurement target can be imaged. Therefore it is preferable to select the visualization target wavelength appropriately, considering a desired resolution and depth. In other words, in the case of the sequential display, an image is reconstructed using acoustic signals that originated from the long wavelength light for a deep region of the object (region in which the propagation length in the object from the light source is long). In the case when the user demands a relatively high resolution even in the sequential display, the image is reconstructed using acoustic signals that originated from light having a wavelength that is characteristically absorbed by the re construction target components. Steps S203 to S212 are the same as steps S103 to S112.

According to this embodiment, when information on substance concentration is acquired using a plurality of wavelengths, the receiving position of each wavelength and the allocation of the receiving positions used for the sequential display can be appropriately determined. As a result, an image display having high followability to the scanning can be implemented.

(Modification)

An example of performing the sequential display using one of the plurality of wavelengths was described above. This method is preferable in terms of displaying a consistent image in the sequential display. However, depending on the arrangement of the signal receiving positions, positions at which light beams with a plurality of wavelengths are received may be included in the visualization targets. Further, consecutive optical pulses having mutually different wavelengths may be regarded as one set. In this case, electric signals are selected in set units. For example, in the case of a two-wavelength set (wavelength 1, wavelength 2), selection becomes “wavelength 1 (first selection), wavelength 2 (first selection), wavelength 1 (second selection), wavelength 2 (second selection) . . . In this case, an image may be reconstructed using the set of the first selection, and not be reconstructed using the set of the second selection. According to this method, oxygen saturation distribution can be displayed even in the sequential display. The sets to be used for reconstruction are selected arbitrarily. For example, an image may be reconstructed using sets of an even number selection.

### Embodiment 3

Embodiment 3 will be described focusing on aspects that are different from the above mentioned embodiments.

(Apparatus Configuration)

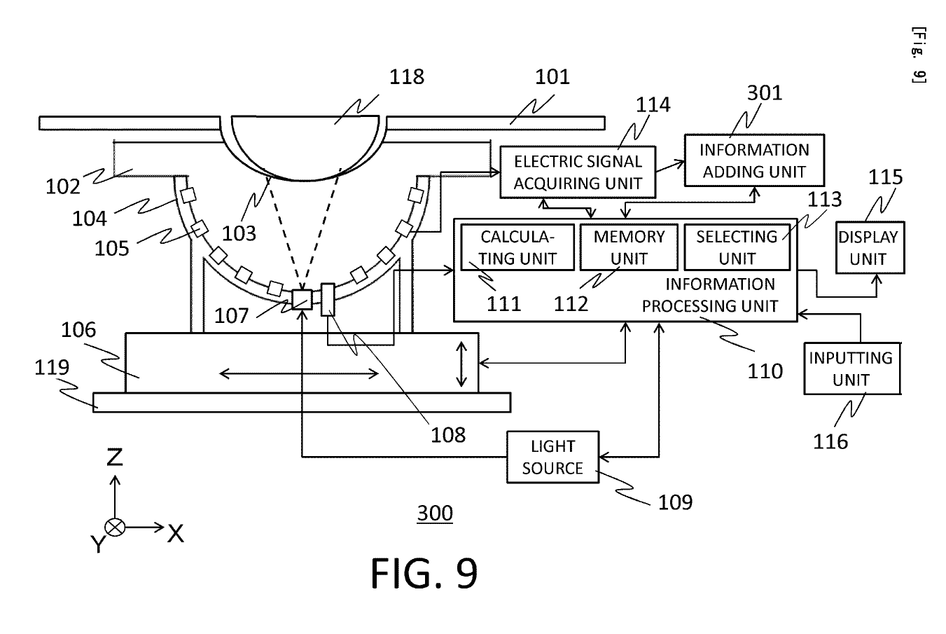

FIG. 9 is a schematic diagram depicting an object information acquiring apparatus 300 according to Embodiment 3. An information adding unit 301 adds information on whether the received signal is the visualization target or not, to the received photoacoustic wave signals acquired by the electric signal acquiring unit 114. For example, bit data which indicates whether the received signal is a visualization target or not is added to the A/D converted received signals. The information adding unit 301 can be constituted by such composing elements as a processing circuit, similarly to the electric signal acquiring unit 114.

(Processing Flow)

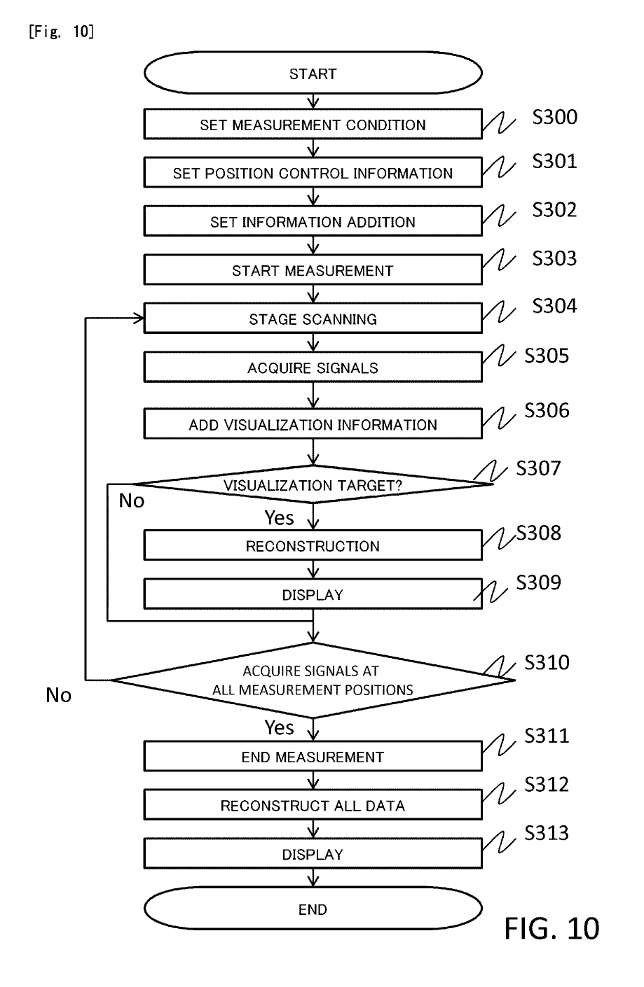

FIG. 10 is a flow chart of the operation according to Embodiment 3.

Steps S300 and S301 are the same as steps S100 and S101 of Embodiment 1.

In step S302, visualization target information, out of the signal received at the photoacoustic wave receiving positions which were set in S301, is set. The calculating unit 111 calculates the visualization target receiving positions based on the measurement conditions, control information, and arrangement information of the plurality of transducers 105 on the supporter 104, which were set in S300 and S301, and performs setting of the information adding unit 301. Or the calculating unit 111 may calculate a number of times of pulsed light irradiation at the visualization target receiving positions, and perform setting for the information adding unit 301. The information on the visualization target receiving positions or information on the number of times of irradiation may be stored in the memory unit 112 in advance. Or the user may input the visualization target receiving positions or the number of times of pulsed light irradiation using the inputting unit 116, and output this information to the information processing unit 110, whereby setting of the information adding unit 301 is performed.

Steps S303 and S304 are the same as steps S103 and S104.

In step S305, light is irradiated by the light source 109, the photoacoustic wave is received by the transducers 105, and signal processing is performed by the electric signal acquiring unit 114, similarly to S105. The plurality of electric signals acquired by the electric signal acquiring unit 114 are output to the information adding unit 301. At this time, the light source transmits a number of times of pulsed light irradiation to the information processing unit 110, and this information is stored in the memory unit 112. Or the information processing unit 110 counts a number of times of pulsed light irradiation, and this information is stored in the memory unit 112.

In step S306, the information adding unit 301 adds information on whether the electric signal is the visualization target or not to the plurality of electric signals acquired in S305 based on the information which was set in S302. For example, the information adding unit 301 determines whether the information is added or not based on the comparison of the coordinate position of the support in S304 and the visualization target receiving position. Or the information adding unit 301 determines whether the information is added or not based on the comparison of the number of times of pulsed light irradiation acquired in S305 and the number of times ∘ pulsed light irradiation which was set. The electric signals to which information on whether this electric signal is the visualization target or not is added are sent to the information processing unit 110, and stored as an electric signal in the coordinate position of the support in S304. These electric signals may also be stored as electric signals associated with the number of times of pulsed light irradiation in S305.

In step S307, it is determined whether or not the signal stored in S306 is a received signal as a signal visualization target of visualization target. For example, the selecting unit 113 reads information added in S306, and if this signal is not the visualization target received signal (NO in S307), processing advances to S310. If this signal is a visualization target received signal (YES in S307), processing advances to S308.

Steps S308 to S313 are the same as steps S107 to S112.

According to Embodiment 3, subsequent selection processing becomes easier by using the information to which the information adding unit 301 added. As a result, the calculation resource can be used for increasing the speed of processing and increasing the resolution, and the sequential display can be more useful. The added information can also be used for a final high definition display.

### Other Embodiments

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2016-021806, filed on Feb. 8, 2016, which is hereby incorporated by reference herein in its entirety.

1. An information acquiring apparatus, comprising:
a calculating unit configured to generate image data, based on signals acquired by transducers receiving an acoustic wave generated from an object by a plurality of times of light irradiation to the object, wherein the plurality of times of light irradiation include light irradiation having a mutually different plurality of wavelengths; and
a display controlling unit configured to cause a display unit to display an image based on the image data,
wherein the calculating unit generates first image data using the signals corresponding to light irradiation having one wavelength of the plurality of wavelengths before the plurality of times of light irradiation complete,
wherein the display controlling unit causes the display unit to display an image based on the first image data before the plurality of times of light irradiation complete,
wherein the calculating unit generates second image data using the signals corresponding to the plurality of times of light irradiation having the plurality of wavelengths after the plurality of times of light irradiation complete, and
wherein the display controlling unit causes the display unit to display an image based on the second image data after the plurality of times of light irradiation complete.

2. The information acquiring apparatus according to claim 1, further comprising a moving unit configured to move the transducers before the plurality of times of light irradiation complete,
wherein the transducers are configured to receive the acoustic wave at a plurality of receiving positions to which the transducers are moved by the moving unit, and
wherein the calculating unit is configured to:
(1) generate the first image data using signals corresponding to partial receiving positions out of the plurality of receiving positions, as the signals corresponding to the part of the plurality of times of light irradiation, and
(2) generate the second image data, using the signals corresponding to a higher number of receiving positions than the partial receiving positions, as the signals corresponding to light irradiation more than the part of the plurality of times of light irradiation.

3. The information acquiring apparatus according to claim 2, further comprising a supporter configured to support the plurality of transducers so as to form a high sensitivity region.

4. The information acquiring apparatus according to claim 3, wherein the calculating unit generates the first image data corresponding to the high sensitivity region.

5. The information acquiring apparatus according to claim 4, wherein when the first image data is displayed, an image corresponding to the high sensitivity region is sequentially added to the displayed image as the position of the transducers change.

6. The image acquiring apparatus according to claim 1, further comprising:
a memory unit configured to store the signal which associates with the light irradiation; and
a selecting unit configured to select the signal corresponding to an acoustic wave generated by predetermined light irradiation, from the signals stored in the memory unit,
wherein the calculating unit generates the image data using the signal selected by the selecting unit.

7. The information acquiring apparatus according to claim 1, further comprising:
a memory unit configured to store the signal which associates with the position of the transducer; and
a selecting unit configured to select the signal from the signals stored in the memory unit, based on the position of the transducer,
wherein the calculating unit generates the image data using the signal selected by the selecting unit.

8. (canceled)

9. The information acquiring apparatus according to claim 1, wherein as the first image data, the calculating unit generates image data that indicates initial sound pressure distribution, optical energy absorption density distribution, or absorption coefficient distribution, and
wherein as the second image data, the calculating unit generates image data that indicates concentration distribution of a substance constituting the object.

10-11. (canceled)

12. The information acquiring apparatus according to claim 1, wherein for each light irradiation having the one wavelength of the plurality of wavelengths, the calculating unit generates the first image data using the signal corresponding to the light irradiation, and
wherein the display controlling unit causes the display unit to display, for each light irradiation having the one wavelength of the plurality of wavelengths, the image based on the first image data.

13. A display method for an image generated based on signals acquired by transducers receiving an acoustic wave generated from an object by a plurality of times of light irradiation to the object, wherein the plurality of times of light irradiation include light irradiation having a mutually different plurality of wavelengths, the display method comprising:
generating first image data using the signals corresponding to light irradiation having one wavelength of the plurality of wavelengths before the plurality of times of light irradiation complete;
causing a display unit to display an image based on the first image data before the plurality of times of light irradiation complete;
generating second image data using the signals corresponding to the plurality of times of light irradiation having the plurality of wavelengths after the plurality of times of light irradiation complete; and
causing the display unit to display an image based on the second image data after the plurality of times of light irradiation complete.

14. A non-transitory storage medium which stores a program causing a computer to execute the display method according to claim 13.

15. An information acquiring apparatus, comprising:
a calculating unit configured to generate image data, based on signals acquired by transducers receiving an acoustic wave generated from an object by a plurality of times of light irradiation to the object, wherein the plurality of times of light irradiation include light irradiation having a mutually different plurality of wavelengths; and
a display controlling unit configured to cause a display unit to display an image based on the image data,
wherein the calculating unit generates first image data that indicates initial sound pressure distribution, optical energy absorption density distribution, or absorption coefficient distribution based on the signals before the plurality of times of light irradiation complete,
wherein the display controlling unit causes the display unit to display an image based on the first image data before the plurality of times of light irradiation complete,
wherein the calculating unit generates second image data that indicates concentration distribution of a substance constituting the object based on the signals after the plurality of times of light irradiation complete, and
wherein the display controlling unit causes the display unit to display an image based on the second image data after the plurality of times of light irradiation complete.

16. A display method for an image generated based on signals acquired by transducers receiving an acoustic wave generated from an object by a plurality of times of light irradiation to the object, wherein the plurality of times of light irradiation include light irradiation having a mutually different plurality of wavelengths, the display method comprising:
generating first image data that indicates initial sound pressure distribution, optical energy absorption density distribution, or absorption coefficient distribution based on the signals before the plurality of times of light irradiation complete;
causing a display unit to display an image based on the first image data before the plurality of times of light irradiation complete;
generating that indicates concentration distribution of a substance constituting the object based on the signals after the plurality of times of light irradiation complete; and
causing the display unit to display an image based on the second image data after the plurality of times of light irradiation complete.

17. A non-transitory storage medium which stores a program causing a computer to execute the display method according to claim 16.

18. The information acquiring apparatus according to claim 9, wherein the concentration distribution includes oxyhemoglobin concentration distribution, deoxyhemoglobin concentration distribution, or oxygen saturation distribution.
